(19) United States
(12) Patent Application Publication
(10) Pub. No.: US 2004/0176310 A1
(43) Pub. Date: Sep. 9, 2004

(54) Use of extracts and preparations from iris plants and tectorigenin as medicaments

(76) Inventors: Wolfgang Wuttke, Bovenden (DE); Hubertus Jarry, Neu-Eichenberg (DE); Michael Popp, Lauf/Pegnitz (DE); Volker Christoffel, Buchberg (DE); Barbara Spengler, Neumarkt (DE)

Correspondence Address: Stephan A Pendorf, Pendorf & Cutliff, 5111 Memorial Highway, Tampa, FL, 33634-7356, US

(21) Appl. No.: 10/477,688

(22) PCT Filed: May 15, 2002
(86) PCT No.: PCT/EP02/05370

(30) Foreign Application Priority Data
May 15, 2001 (DE) 101 23 503.8

(51) Int. Cl. A61K 31/704; A61K 35/78

(52) U.S. Cl. 514/26; 424/725

(57) ABSTRACT

The present invention relates to extracts from the lridaceae plant Belamcanda chinensis for producing a ready-formulated medicament suited for the selective treatment and/or prophylaxis of sexual hormone-related disorders of the uro-genital tract. The inventive extracts are moreover suited for the treatment and/or prophylaxis of post-menopausal bladder infections and of urinary incontinence. The are moreover indicated for the treatment of benign and malignant prostate hyperplasies. It was moreover found that the ingredient tectorigenin as well as its glycosides, isolated from Belamcanda chinensis, essentially has the same effects as the whole extract.

The present invention relates to a use of extracts from iridaceae of the genus Belamcanda chinensis in accordance with claims 1 and 3. It moreover relates to a use of tectorigenin and/or tectorigenin glycosides and/or extracts containing tectorigenin and/or tectorigenin glycoside(s) in accordance with claims 4 and 6.

It is known from WO99/47 149 (=EP1064009A1) to the applicant of the present application that extracts from Belamcanda chinensis and of the isoflavonoid tectorigenin contained therein have an estrogenic effect not on the uterus, however in the hypothalamo-hypophysary axis, in the cardiovascular system, and in the bone.

17β-estradiol, which is formed in women's ovaries (any mention of estradiol hereinbelow refers to physiological 17β-estradiol) [hereinafter also referred to as E₂], has a generally proliferation-enhancing effect in the organs. In addition to controlling the female cycle, it i.a. also has a homeostatic effect on the functions of urethra, bladder and vagina. In men, locally formed estradiol as well as the male sexual hormone testosterone also have a proliferation-enhancing effect in the bladder and in the prostate.

During menopause, lowering of the estradiol level takes place due to cessation of the ovarial function. This results in weakening of proliferative processes in many organs, with subsequently occurring degeneration and reduced functional capacity.

With lack of estrogen, cystitis frequently develops in the post-menopausal woman as a consequence of reduced mucous membrane in the urethra and thus facilitated ascension of germs.

Urinary incontinence frequently occurring post-menopausally is equally intensified by lack of estrogen. These disorders may be prevented or alleviated, respectively, by timely administration of estradiol.

Hormonal regulation of the function of the bladder in men presumably is similar to that in women; in any case the hitherto known regulative principles in the bladder of male and female rats are very similar.

The administration of estrogens having a nonselective proliferative action is particularly adverse for uterus and breast tissue, for uncontrolled growth may occur here owing to the insufficiency or lack of gestagen counter-regulation.

Up to the present, there is no medicament available from plant drugs that might be used for an organoselective prophylaxis or therapy in cases of estrogen deficiency.

Starting out from this prior art, it accordingly is an object of the present invention to furnish plant medicaments having a sexual hormone-type action while having an organselective action with no effect or only little effect on the uterus.

This object is achieved through the characterizing features of claims 1, 3, 4 and 6.

In particular, the present invention relates to

use of extracts from Belamcanda chinensis for producing an estrogen-type, organoselective medicament having no uterotrophic effect or one that is at least negligible, wherein

the medicament is used for alleviating sexual hormone-related disorders of the uro-genital tract.

Use encompassing the postmenopausally occurring disorders encompass atony of the bladder (urinary incontinence), cystitis, and dry vagina is preferred

In addition the present invention relates to a use of extracts from Belamcanda chinensis for producing an estrogen-type, organoselective medicament having no uterotrophic effect or one that is at least negligible, wherein

the medicament is used for alleviating benign and malignant hyperplasy of the prostate.

The present invention also relates to a use of tectorigenin and/or its glycosides and/or extracts containing tectorigenin and/or tectorigenin glycoside(s) for producing an estrogen-type, organoselective medicament having no uterotrophic effect or one that is at least negligible, wherein

the medicament is used for alleviating sexual hormone-related disorders of the uro-genital tract.

Particularly preferred is the use of tectorigenin, the derivatives thereof, in particular its glycosides, as well as extracts containing tectorigenin and/or tectorigenin glycoside(s) for treating or alleviating, respectively, the postmenopausally occurring disorders atony of the bladder (urinary incontinence), cystitis, and dry vagina.

Use of tectorigenin and/or its glycosides and/or extracts containing tectorigenin and/or tectorigenin glycoside(s) for producing an estrogen-type, organoselective medicament having no uterotrophic effect or one that is at least negligible, wherein

the medicament is used for alleviating benign and malignant hyperplasy of the prostate.

It was surprisingly found that Belamcanda chinensis extract also has effects similar to those of estradiol in the bladder and in the urethra of ovarectomized rats. In the bladder of castrated rats, estradiol and Belamcanda analogously regulate down the gene expression of the estrogen receptor-alpha.

Bladder tonus is largely influenced by nerve cells located in the bladder wall and other vegetative nerve cells. The “Nerve Growth Factor” (NGF) is an indicator for the local activity of the nerve cells. Surprisingly the gene expression of this growth factor is highly significantly stimulated by Belamcanda—an effect observed under neither estradiol nor testosterone.

A desirable effect of the extract on innervation of the bladder and thus an improvement of urinary incontinence is held to be secured.

Benign prostatic hyperplasy and the prostate carcinoma in men may also be influenced favorably by inhibition of the local estrogen production, but also by inhibiting synthesis of the male sexual hormones.

The like active mechanisms may also be demonstrated on the male castrated rat for the Belamcanda chinensis extract. Estradiol and the Belamcanda extract significantly inhibit gene expression of the estrogen receptor of alpha-subtype in the prostate without having an effect on the gene expression of the estrogen receptor-beta.

Both in in-vivo experiments in the rat prostate in the whole-body model and in human prostate carcinoma cells it was surprisingly found that Belamcanda chinensis extracts and extracts containing tectorigenin, as well as the pure substance itself, have an anti-androgenic action favorably influencing prostate carcinoma and significantly inhibiting growth of the tumor cells in vitro.

In the prostate of castrated male rats, gene expression of the proliferation-enhancing growth factor IGF1 is moreover regulated up by testosterone, however regulated down by Belamcanda, as is desired for reducing the hyperplasy. The prostate regression induced by castration could be antagonized by testosterone in experiments with rats. In analogy with the findings in the case of castrated rats not treated with androgen (see above), the IGF1 expression regulated up by testosterone (see above) could also be regulated down by the Belamcanda extract. These findings clearly demonstrate that the testosterone-induced prostate growth may be inhibited by Belamcanda chinensis extract.

In experiments on the human prostate-Ca line LNCAP, 5 alpha-dihydrotestosterone-induced proliferation and the production of its prostate-specific antigen (PSA) may significantly be inhibited by Belamcanda chinensis extract.

Inasmuch as IGF1 always participates in the proliferation not only of benign but also malignant tumors, the inhibitory effect of Belamcanda chinensis extract on IGF1-gene expression in conjunction with this finding is evidence for an inhibitory effect on the prostate carcinoma.

As regards manufacture of the extract in accordance with the invention, WO99/47 149 is herewith fully included herein by reference, the essential features of which are, however, presently summarized as follows:

It was found through in-vitro and in-vivo experimentation that extracts produced both from Iridaceae, in particular Belamcanda chinensis, with organic solvents or mixtures of organic solvents with water or with supercritical CO₂ have an organselective effect on the uro-genital system, with an effect on the uterus being absent.

Thus the extracts used in accordance with the invention are suited for producing a ready-formulated medicament for the selective treatment and/or prophylaxis of sexual hormone-related disorders of the uro-genital tract.

Furthermore they are suitable for producing a ready-formulated medicament for the selective treatment and/or prophylaxis of post-menopausal bladder infections and of urinary incontinence.

They are moreover suited for producing a ready-formulated medicament for the treatment of benign and malignant prostate hyperplasies.

It was moreover found that the ingredient tectorigenin that was isolated from Belamcanda chinensis essentially has the same effects as the whole extract.

Apart from Belamcanda chinensis, this ingredient is also found in other iridaceae such as, e.g., Iris germanica, I. tectorum, I. illyrica, I. dichotoma. Preferably rhizomes, stalks, leaves and/or petals of the plants are used for producing the extracts.

1. Use of extracts from Belamcanda chinensis for producing an estrogen-type, organoselective medicament having no uterotrophic effect or one that is at least negligible,
characterized in that
the medicament is used for alleviating sexual hormone-related, post-menopausally occurring disorders of the uro-genital tract.

2. Use in accordance with claim 1, characterized in that the postmenopausally occurring disorders atony of the bladder (urinary incontinence), postmenopausally occurring cystitis, and dry vagina are encompassed.

3. Use of extracts from Belamcanda chinensis for producing an estrogen-type, organoselective medicament,
characterized in that
the medicament is used for alleviating benign and malignant hyperplasy of the prostate.

4. Use of tectorigenin and/or its glycosides and/or extracts containing tectorigenin and/or tectorigenin glycoside(s) for producing an estrogen-type, organoselective medicament,
characterized in that
the medicament is used for alleviating sexual hormone-related, Post-menopausally occurring disorders of the uro-genital tract.

5. Use in accordance with claim 4, characterized in that the postmenopausally occurring disorders atony of the bladder (urinary incontinence), cystitis, and dry vagina are encompassed.

6. Use of tectorigenin and/or its glycosides and/or extracts containing tectorigenin and/or tectorigenin glycoside(s) for producing an estrogen-type, organoselective medicament having no uterotrophic effect or one that is at least negligible,
characterized in that
the medicament is used for alleviating benign and malignant hyperplasy of the prostate.
